(19) United States
(12) Patent Application Publication
(10) Pub. No.: US 2024/0416024 A1
(43) Pub. Date: Dec. 19, 2024

(54) ENDOLUMINAL TREATMENT DEVICES AND RELATED METHODS

(71) Applicant: Boston Scientific Scimed, Inc., Maple Grove, MN (US)

(72) Inventors: Paul SMITH, Smithfield, RI (US); Rahul PRABHU, Cambridge, MA (US); Jonathan ROOT, Townsend, MA (US); Scott E. BRECHBIEL, Burlington, MA (US)

(73) Assignee: Boston Scientific Scimed, Inc., Maple Grove, MN (US)

(21) Appl. No.: 18/741,959

(22) Filed: Jun. 13, 2024

Related U.S. Application Data
(60) Provisional application No. 63/508,032, filed on Jun. 14, 2023.

(51) Int. Cl. A61M 1/00; A61B 1/00

(52) U.S. Cl. CPC A61M 1/915; A61B 1/00094

(57) ABSTRACT

A medical device may include an implant having a plurality of openings, a tube having a lumen extending therethrough, a body extending from a distal portion of the tube to a proximal portion of the implant, such that the plurality of openings are in fluid communication with the lumen, and a grasping portion configured to be grasped by a second medical device.

### CROSS-REFERENCE TO RELATED APPLICATIONS

This application claims the benefit of priority to U.S. Provisional Application No. 63/508,032, filed on Jun. 14, 2023, which is incorporated by reference herein in its entirety.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Patients with perforations, post-surgical leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices of the current disclosure may rectify some of the deficiencies described above or address other aspects of the art.

### SUMMARY

Each of the aspects disclosed herein may include one or more aspects of the features described in connection with any of the other disclosed aspects.

In an aspect, a medical device may include an implant having a plurality of openings, a tube having a lumen extending therethrough, and a body extending from a distal portion of the tube to a proximal portion of the implant, such that the openings may be in fluid communication with the lumen. The medical device may further include a grasping portion configured to be grasped by a second medical device.

Any of the aspects disclosed herein may include any of the following features, alone or in any combination. The grasping portion may include at least one of a tab, a strap, or an opening. The tube may be removably coupled to the body. A distal portion of the body may be wider than a proximal portion of the body. A distal opening of the body may be configured to receive the proximal portion of the implant and a proximal opening of the body may be configured to receive the distal portion of the tube. A first portion of the distal opening may be distal to a second portion of the distal opening. The body may include a lumen extending from the distal opening to the proximal opening. The proximal portion of the implant may be compressed within the body. The tube may be configured to couple to a vacuum source to supply a negative pressure to the implant. The grasping portion may include at least one opening formed on a radially outer surface of the body. The at least one opening may be positioned adjacent to a distal opening of the body. The grasping portion may include at least one strap fixed to a radially outer surface of the body. The grasping portion may include at least one tab extending radially outward from the implant. A protruding portion of the at least one tab may extend radially beyond a radially outer surface of the implant. The implant may be disposed over a portion of the body, and the grasping portion may include a ring positioned over the implant and the body. The grasping portion may include a tab extending from the ring.

In another example, a medical device may include an implant having a plurality of openings, a tube having a lumen extending therethrough and in fluid communication with the plurality of openings, a body extending from a proximal end to a distal end, and a grasping portion configured to be grasped by a second medical device. The body may taper radially inward from the distal end to the proximal end and may couple the implant to the tube

Any of the aspects disclosed herein may include any of the following features, alone or in any combination. The distal end of the body may be angled. A first portion of a distal opening of the body may be distal to a second portion of the distal opening of the body. The grasping portion may include an opening formed on a radially outer surface of the body. The opening may be adjacent to an outer edge of the distal opening.

In a further aspect, a medical device may include an implant having a plurality of openings and a tube having a lumen extending therethrough. The tube may be configured to couple to a vacuum source. The medical device may further include a body coupling the implant to the tube, such that the plurality of openings may be in fluid communication with the lumen, and a grasping portion configured to be grasped by a second medical device. The grasping portion may include at least one of a tab, a strap, or an opening. The body may include the grasping portion.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIGS. 1A and 1B illustrate an exemplary medical device, according to aspects of this disclosure.

FIG. 2 illustrates another exemplary medical device, according to aspects of this disclosure.

FIG. 3 illustrates a further exemplary medical device, according to aspects of this disclosure.

FIG. 4 illustrates another exemplary medical device, according to aspects of this disclosure.

FIGS. 5A-5D illustrate a further exemplary medical device, according to aspects of this disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge/foam (e.g., vacuum sealed foam) at its distal end. A proximal end of the tube may be connected to a collection container. The sponge is placed endoscopically into the perforation, leak, or other wound. In some examples, EVAC includes endoluminal placement of a sponge or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., the colon). Negative pressure then is applied.

The foam in the wound, along with the negative pressure, may accelerate healing by encouraging local tissue granulation at a wound site. The foam may be replaced with increasing smaller sizes of foam as the wound heals and closes. Devices and systems suited for EVAC are limited. During EVAC, a foam may be delivered to a target site using a scope, rat-tooth forceps, and a suture loop at a distal end of the foam. For example, the rat-tooth forceps may be extended through a working channel of the scope and used to grasp the loop at the distal end of the foam. The rat-tooth forceps may be used to move the foam to a target site as the scope is navigated to the target site, which can present challenges. For example, the foam may block scope visualization as a user moves the foam through the GI tract using the rat-tooth forceps. Furthermore, a suture loop may be useful for initial placement of a foam near the target site, but not for positioning of the foam at or in the target site due to the non-rigid structure of the suture loop. For example, a suture loop may not be useful in positioning a foam at difficult target sites, e.g., cardia regions of a sleeve gastrectomy.

Removal of a foam from a target site during EVAC also presents challenges. For example, tissue may grow into the foam if left at the target site for an extended period of time, which may make it difficult to remove the foam from the target site during, e.g., foam replacement. As such, EVAC typically requires a foam to be replaced every 3 to 5 days to prevent ingrowth and to maintain cleanliness of the target site.

Aspects of this disclosure include devices and methods to improve delivery of an implant (e.g., a foam) to a target site and/or implant replacements during EVAC. For example, aspects of this disclosure include devices and methods to reduce the time needed to deliver an implant (e.g., a foam) to a target site and/or reduce the time needed to remove an implant from a target site. For example, aspects of this disclosure may improve implant (e.g., foam) exchanges at a target site.

Components of the devices described herein may be packaged as a kit for EVAC. For example, the devices disclosed herein may include a connector coupled to a proximal end of a foam. In some aspects, the connector may be configured to couple a vacuum tube with the foam to treat a wound. In some aspects, the connector may form a rigid coupling with the vacuum tube and the foam. In some aspects, the connector may include openings or loops configured to receive distal ends of tools. In some examples, the connector may allow for quick foam exchanges during an EVAC procedure. In some aspects, the connector may allow for improved visualization of the foam as the foam is delivered to a target site.

Reference will now be made in detail to examples of the present disclosure described above and illustrated in the accompanying drawings. Wherever possible, the same reference numbers will be used throughout the drawings to refer to the same or like parts.

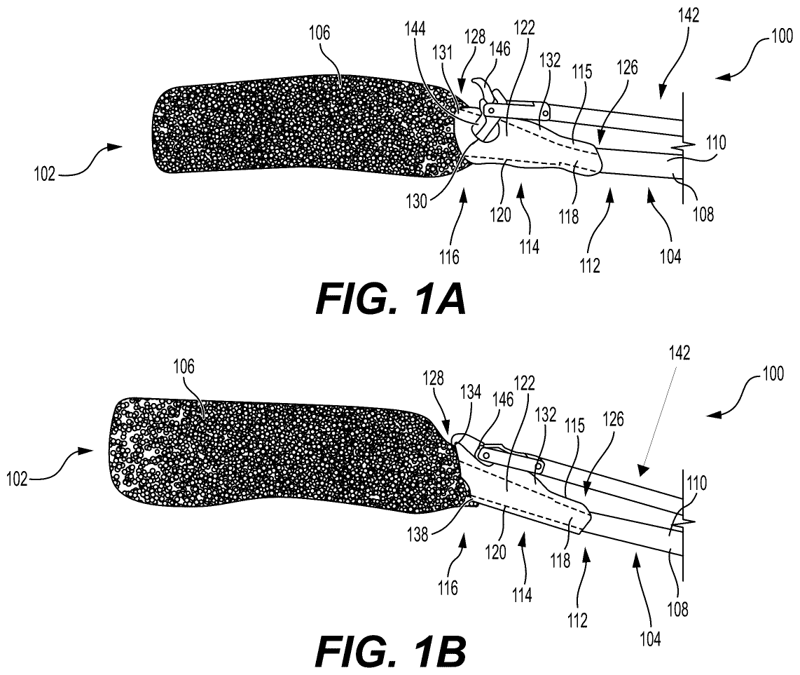

FIGS. 1A and 1B illustrate a distal end of an exemplary EVAC device 100 that may be inserted into a patient for treatment of wounds or otherwise used to help treat tissue. EVAC device 100 may include a foam 102 (an implant), such as a sponge or other porous body, a vacuum tube 104 or other type of conduit, and a connector 114. Connector 114 may include a body 115 extending from a distal portion 112 of vacuum tube 104 to a proximal portion 116 of foam 102. Connector 114 may be at least one of a tab, ring, or pocket extending from foam 102 and/or vacuum tube 104. Connector 114 will be described in greater detail below.

Foam 102 may have any features of any foam that is known in the art for use in EVAC procedures. For example, foam 102 may include an open-cell foam. Foam 102 may include openings 106 on an outer surface and/or interior thereof. Openings 106 may be any hole, pore, or channel. Openings 106 may include interconnecting channels and/or pores throughout foam 102. For example, openings 106 may be pores of foam 102. Openings 106 may have different sizes and/or shapes. Features of openings 106 (e.g., a size and shape of pores of foam 102) may be selected based on a location of treatment within the body, properties of a wound to be treated, a stage of treatment, or other factors. Foam 102 is illustrated as having a cylindrical shape, but may be any shape, including spherical, cuboidal, irregular, or the like. Foam 102 may be compressed into a lower profile during insertion to a target site and may expand during deployment at a target site.

Foam 102 may include any suitable biocompatible material that may absorb liquids and/or permit liquid or other materials to pass therethrough via, e.g., negative pressure applied to foam 102. The material of foam 102 may be flexible, compressible, porous, hydrophilic, sterile, and/or disposable. Suitable materials include polyurethanes, esters, ethers, composite materials, and/or other medical-grade materials.

Vacuum tube 104 may include an outer wall 108 defining one or more lumens 110. Lumen 110 may be open at both a proximal end (not shown) and a distal portion 112 of vacuum tube 104 within or otherwise coupled to foam 102. Openings 106 of foam 102 may be in fluid communication with the distal opening of vacuum tube 104. The proximal end of vacuum tube 104 may be connected to a vacuum source (not shown), which may supply a negative pressure to foam 102, via a distal opening of vacuum tube 104. For example, a negative pressure of approximately 125 mm Hg, or approximately 2.5 pounds per square inch (PSI), may be supplied to foam 102 via vacuum tube 104. Other suitable amounts of negative pressure may be used. The negative pressure may pull fluid, material, and/or other debris into lumen 110 of vacuum tube 104 via openings 106 of foam 102, which may promote healing of a target site.

As mentioned above, connector 114 may include body 115 extending from distal portion 112 of vacuum tube 104 to proximal portion 116 of foam 102. Body 115 of connector 114 may include radially outer surface 132, a proximal opening 126 at a proximalmost end of body 115, and a distal opening 128 at a distalmost end of body 115. A lumen 120 (illustrated with dashed lines to show an internal feature of body 115) may extend through body 115 from proximal opening 126 to distal opening 128. A distalmost end of body 115 may be tapered or angled, such that a first portion 134 of distal opening 128 is distal to a second portion 138 of distal opening 128 (shown in FIG. 1B). In other words, a tip of body 115 may have a beveled shape, such that distal opening 128 has an elongated shape. In alternative examples, distal opening 128 of connector 114 may be substantially perpendicular to a longitudinal axis of connector 114, or may be in any other suitable shape, size, and/or orientation.

Body 115 of connector 114 may taper radially inward from its distalmost end to its proximalmost end. A distal portion 122 of lumen 120 may be wider than a proximal portion 118 of lumen 120. Proximal portion 118 of lumen 120 may be configured to receive distal portion 112 of vacuum tube 104. For example, distal portion 112 of vacuum tube 104 may extend distally through proximal opening 126 and through proximal portion 118 of lumen 120 of body 115. Inner walls of proximal portion 118 of lumen 120 may contact portions of outer wall 108 of distal portion 112 of vacuum tube 104 to tightly secure connector 114 to distal portion 112 of vacuum tube 104. In some examples, connector 114 may be fixed to distal portion 112 of vacuum tube 104. In some examples, connector 114 may be of a heat shrinkable or moldable material formed over outer wall 108 of distal portion 112 of vacuum tube 104 and/or proximal portion 116 of foam 102, as discussed below. In some examples, distal portion 112 of vacuum tube 104 may further extend through distal portion 122 of lumen 120.

Distal portion 122 of lumen 120 may be configured to receive proximal portion 116 of foam 102. For example, distal portion 122 of lumen 120 may form a pocket or cavity that may be sized and shaped to receive proximal portion 116 of foam 102. Proximal portion 116 of foam 102 may be compressed when placed within distal portion 122 of lumen 120. Alternatively, a proximal portion 116 of foam 102 may have a smaller diameter/width than more distal portions of foam 102 in a natural/relaxed configuration. In an assembled configuration, proximal portion 116 of foam 102 may be positioned adjacent to distal portion 112 of vacuum tube 104 within lumen 120. For example, distal portion 112 of vacuum tube 104 may extend into proximal portion 116 of foam 102. Distal portion 112 of vacuum tube 104 may be attached to foam 102 via sutures or other ties, an adhesive, a shrink-wrapped material, elastic(s) or the like. In one example, a recess or neck (not shown) may be provided in foam 102 (e.g., proximal portion 116 of foam 102) to receive distal portion 112 of vacuum tube 104.

Body 115 of connector 114 may have a shape that is similar to a strain relief portion. Body 115 may have a tapered shape, such that it extends gradually radially outward from a width/diameter similar to a width/diameter of vacuum tube 104 to a width/diameter similar to a width/diameter similar to proximal portion 116 of foam 102. In other words, connector 114 may transition between vacuum tube 104 and foam 102.

In some examples, body 115 of connector 114 may be rigid, such that body 115 may maintain its original form or shape when grasped by, e.g., a tool. Connector 114 may be rigidly coupled to foam 102 and vacuum tube 104, such that movement and/or manipulation of connector 114 results in movement and/or manipulation of foam 102 and vacuum tube 104 approximately one to one. Furthermore, a rigid coupling between connector 114 and foam 102 (and vacuum tube 104) may allow for transfer of directional forces to move and/or manipulate the foam 102 to a target site. In alternatives, body 115 may be flexible.

Body 115 may include a side opening 130 formed in radially outer surface 132 and in communication with lumen 120. Side opening 130 may be disposed adjacent to an outer edge of first portion 134 of distal opening 128. In alternatives, side opening 130 may be disposed on any portion of radially outer surface 132. Side opening 130 may be configured to receive a grasper 142, or any suitable medical tool, e.g., an extending rod, a hook, or the like, to deliver foam 102 to a target site. Side opening 130 may form a tag 131 (labeled in FIG. 1A) between a distal end of opening 130 and a distalmost edge of body 115. Grasper 142 may grasp tag 131. Side opening 130 may be any suitable size and/or shape (e.g., circular or ovular). For example, a first arm 144 of grasper 142 may extend through side opening 130 and a second arm 146 of grasper 142 may be closed over radially outer surface 132 to grasp connector 114. As shown in FIGS. 1A and 1B, distal ends of arms 144, 146 may be coupled to one another adjacent the outer edge of first portion 134 of distal opening 128. The arrangement of opening 130 and grasper 142 in FIGS. 1A and 1B is merely exemplary. In alternatives, tag 131 may be proximal of opening 130. Grasper 142 or another tool may face radially outward, distally, and/or proximally when grasping tag 131.

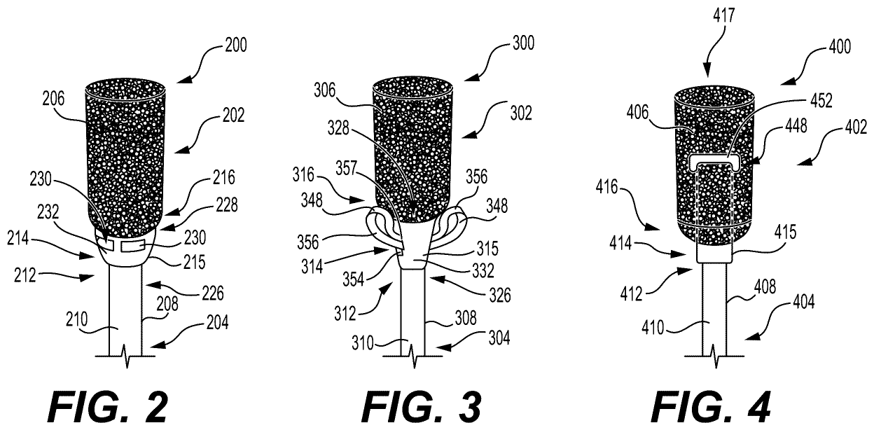

FIG. 2 illustrates a distal end of another exemplary EVAC device 200. EVAC device 200 may have similar properties to EVAC device 100, unless stated otherwise herein. Where feasible, 100 has been added to reference numbers related to EVAC device 100 similar to aspects of EVAC device 200. EVAC device 200 may include a foam 202 (an implant), such as a sponge or other porous body, a vacuum tube 204, and a connector 214. Similar to vacuum tube 104, vacuum tube 204 may include an outer wall 208, a lumen 210, and a proximal end of vacuum tube 204 may be connected to a vacuum source (not shown). Openings 206 of foam 202 may be in fluid communication with lumen 210 of vacuum tube 204, such that when negative pressure is supplied to lumen 210, the negative pressure may pull fluid, material, and/or other debris from a target site into lumen 210 of vacuum tube 204 via openings 206 or foam 202.

Similar to connector 114, connector 214 may include a body 215 extending from a distal portion 212 of vacuum tube 204 to a proximal portion 216 of foam 202. Body 215 may include a radially outer surface 232, a proximal opening 226, and a distal opening 228. A lumen (not shown in FIG. 2 and having any of the properties of lumen 120) may extend through body 215 from proximal opening 226 to distal opening 228. As with lumen 120, a distal portion of the lumen of body 215 may be wider than a proximal portion of the lumen of body 215. The proximal portion of the lumen of body 215 may be configured to receive distal portion 212 of vacuum tube 204 and the distal portion of the lumen of body 215 may be configured to receive proximal portion 216 of foam 202. Foam 202, vacuum tube 204, and connector 214 may be coupled to one another and/or assembled in the same or similar manner as described above for EVAC device 100.

Body 215 of connector 214 may include a plurality of side openings 230 on radially outward surface 232 and in communication with the lumen of body 215. Similar to body 115, body 215 may be rigid. In some examples, body 215 may be made from a rigid elastomer. Similar to side opening 130, side openings 230 may be any suitable size and/or shape configured to receive a distal end of a suitable medical tool, such as grasper 142 (FIGS. 1A-1B), an extending rod, a hook, or the like. Side openings 230 may allow for grasper 142 to securely latch onto connector 214 from multiple sides of connector 214 (wherever one of side openings 230 is disposed) and deliver foam 202 to a target site. The plurality of side openings 230 may allow for one or more tool(s) to securely grasp body 215 of connector 214 at different portions of body 215 at the same time.

FIG. 3 illustrates a distal end of another exemplary EVAC device 300. EVAC device 300 may have similar properties to EVAC devices 100 and/or 200, unless stated otherwise herein. Where feasible, 200 has been added to reference numbers related to EVAC device 100 to similar aspects of EVAC device 300. EVAC device 300 may include a foam 302 (i.e., an implant), such as a sponge or other porous body, a vacuum tube 304, and a connector 314. Similar to vacuum tube 104, vacuum tube 304 may include an outer wall 308, a lumen 310, and a proximal end of vacuum tube 304 may be connected to a vacuum source (not shown). Openings 306 of foam 302 may be in fluid communication with lumen 310 of vacuum tube 304, such that when negative pressure is supplied to lumen 310, the negative pressure may pull fluid, material, and/or other debris from a target site into lumen 310 of vacuum tube 304 via openings 306 of foam 302.

Similar to connector 114, connector 314 may include a body 315 extending from a distal portion 312 of vacuum tube 304 to a proximal portion 316 of foam 302. Body 315 may include a radially outer surface 332, a proximal opening 326, and a distal opening 328. A lumen having any of the properties of lumen 120 may extend through body 315 from proximal opening 326 to distal opening 328. A distal portion of the lumen of body 315 may be wider than a proximal portion of the lumen of body 315. The proximal portion of the lumen of body 315 may be configured to receive distal portion 312 of vacuum tube 304, and the distal portion of the lumen of body 315 may be configured to receive proximal portion 316 of foam 302. Foam 302, vacuum tube 304, and connector 314 may be coupled to one another and/or assembled in the same or similar manner as described above for EVAC device 100. Similar to body 215, body 315 may be rigid (e.g., made of a rigid elastomer) or may include any alternative suitable material.

Body 315 of connector 314 may include a plurality of straps 348 disposed on radially outer surface 332. FIG. 3 illustrates two straps 348, however, in some examples, any number of straps 348 may be disposed on radially outer surface 332. A first end 354 of strap(s) 348 may be fixed to radially outer surface 332 and a looped portion 356 of straps(s) 348 may extend radially outward. A second end 357 of strap(s) 348 may be fixed to radially outer surface 332. For example, second end 357 may be fixed distally to first end 354. Straps(s) 348 may be any suitable size and/or shape configured to receive a distal end of a suitable medical tool, such as grasper 142, an extending rod, a hook, or the like. Straps(s) 348 may allow for grasper 142 to securely latch onto connector 314 from any side of connector 314, and deliver foam 302 to a target site. Straps(s) 348 may be elastic or inelastic and may be made of any suitable material.

FIG. 4 illustrates a distal end of another exemplary EVAC device 400. EVAC device 400 may have similar properties to EVAC device 100, unless stated otherwise herein. Where feasible, 300 has been added to reference numbers related to EVAC device 100 similar to aspects of EVAC device 400. EVAC device 400 may include a foam 402 (i.e., an implant), such as a sponge or other porous body, a vacuum tube 404, and a connector 414. Similar to vacuum tube 104, vacuum tube 404 may include an outer wall 408, a lumen 410, and a proximal end of vacuum tube 404 may be connected to a vacuum source (not shown). Openings 406 of foam 402 may be in fluid communication with lumen 410 of vacuum tube 404, such that when negative pressure is supplied to lumen 410, the negative pressure may pull fluid, material, and/or other debris from a target site into lumen 410 of vacuum tube 404 via openings 406.

Similar to connector 114, connector 414 may include a body 415 extending from a distal portion 412 of vacuum tube 404 to a proximal portion 416 of foam 402. As shown in FIG. 4, body 415 may be relatively shorter along a longitudinal/axial direction than bodies 115, 215, 315. As shown in FIG. 4, distal portion 412 of vacuum tube 404 may extend into foam 402 (e.g., into a recess formed within foam 402). EVAC devices 100, 200, and 300 may also have a similar arrangement. In some examples, foam 402, vacuum tube 404, and connector 414 may be coupled to one another and/or assembled in the same or similar manner as described above for EVAC device 100.

EVAC device 400 may further include a tab 448 extending radially outward from a side of foam 402 (e.g., a portion of foam 402 between proximal portion 416 of foam 202 and a distal portion 417 of foam 202). For example, tab 448 may be disposed approximately at a middle of foam 402 in an axial direction. In aspects, tab 448 may be disposed at or near a distalmost end of vacuum tube 404. In some examples, tab 448 may be coupled to vacuum tube 404, and tab 448 may extend through a side wall of foam 402. Alternatively, tab 448 may be disposed at any suitable alternative location along an axial length of foam 402. Although one tab 448 is depicted, it will be appreciated that EVAC device 400 may include any suitable number of tabs, in any suitable location. For example, multiple tabs 448 may be disposed at different axial and/or radial locations along foam 402. A protruding portion 452 of tab 448 may extend radially beyond a radially outer surface of foam 402.

Tab 448 and connector 414 may be rigid and/or made of a rigid elastomer. Alternatively, tab 448 may be flexible (elastic or inelastic). Tab 448 may be any suitable size and/or shape configured to receive a distal end of a suitable medical tool, such as grasper 142, an extending rod, a hook, or the like. Tab 448 may allow for grasper 142 to be securely coupled to foam 402 and/or vacuum tube 404 to deliver foam 402 to a target site.

It will be appreciated that bodies 115, 215, 315, 415 of connectors 114, 214, 314, 414 may include at least one of and/or any combination of side openings 130, 230, strap(s) 348, or tab(s) 448, described above, to facilitate delivery of foams 102, 202, 302, and 402 to a target site. Side openings 130, 230, straps(s) 348, and/or tabs 448 may be positioned in multiple different areas of bodies 115, 215, 315, 415 and oriented in any direction, including facing outward, backward/proximally, or forward/distally. This may facilitate easy grasping or latching on of the connectors 114, 214, 314, 414 by a tool.

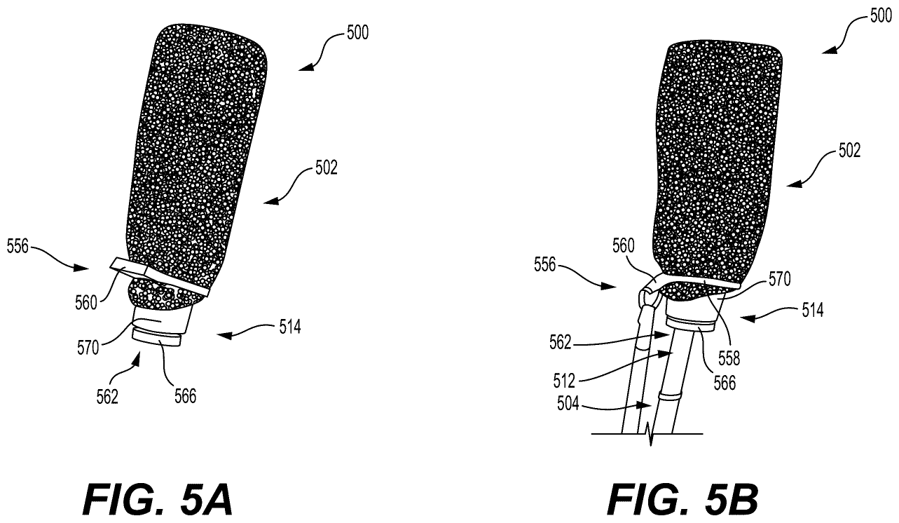

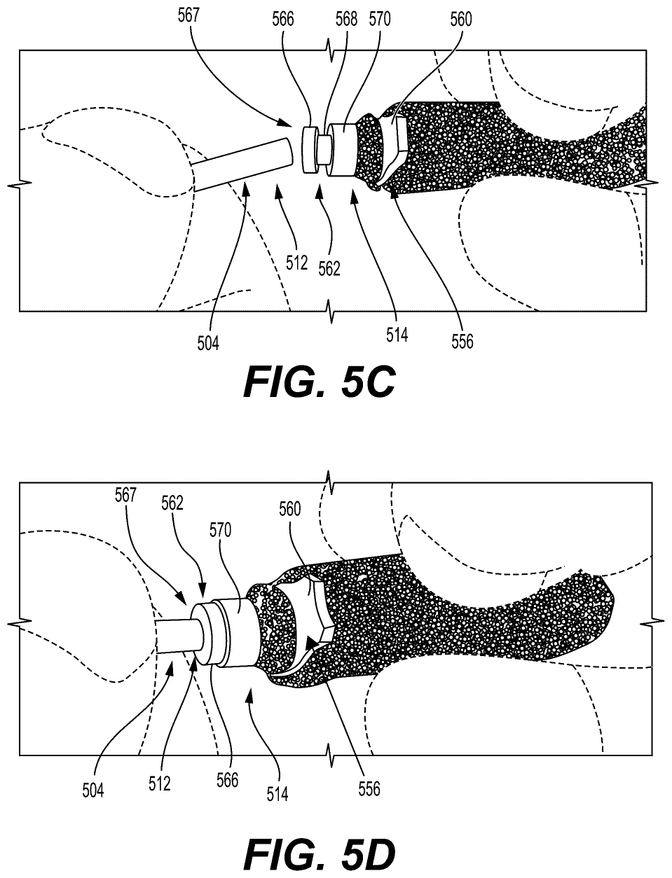

FIGS. 5A-5D illustrate a distal end of another exemplary EVAC device 500. EVAC device 500 may have similar properties to EVAC device 100, unless stated otherwise herein. Where feasible, 400 has been added to reference numbers related to EVAC device 100 similar to aspects of EVAC device 500. EVAC device 500 may include a foam 502 (an implant), such as a sponge or other porous body and a vacuum tube 504. Similar to vacuum tube 104, vacuum tube 504 may include an outer wall and a lumen. A proximal end of vacuum tube 504 may be connected to a vacuum source (not shown). Openings of foam 502 (having any of the properties of the openings discussed above) may be in fluid communication with the lumen of vacuum tube 504, such that when negative pressure is supplied to the lumen, the negative pressure may pull fluid, material, and/or other debris from a target site into the lumen of vacuum tube 504 via the openings of foam 502.

Connector 514 of EVAC device 500 may be configured to improve foam 502 replacements during an EVAC procedure. For example, connector 514 may allow for easy replacement of a larger sized foam 502 for a smaller sized foam 502 at a target site, as a wound at a target site heals. A distal end of connector 514 may be positioned within a recess (not shown) of foam 502.

In some examples, connector 514 may include a collet 562 and a housing member 570. In some examples, foam 502 may be disposed over housing member 570 of connector 514. Collet 562 may be received within a lumen of housing member 570. Collet 562 may have any suitable configuration and may be configured to releasably retain vacuum tube 504. Connector 514 may also include seals (e.g., O-rings) or other structures within housing member 570. Connector 514 may have alternative arrangements that are configured to releasably retain vacuum tube 504. Connector 514 may be similar to a quick connect fitting or push-to-connect fitting and may have properties of any quick connect fitting or push-to-connect fitting known in the art.

As discussed above, connector 514 may include collet 562. Collet 562 may include a flange 566 and a stem 568 (see FIG. 5C). Stem 568 may be received within housing member 570. Flange 566 may be disposed proximally of housing member 570. Collet 562 may be movable in a proximal direction and/or a distal direction relative to housing member 570. In some examples, flange 566 may define a stop, preventing collet 562 from moving distally past a distal wall of flange 566.

Flange 566 may define an opening 567 (see FIGS. 5C and 5D) for receiving a distal end of vacuum tube 504. Opening 567 may be a proximal opening of a lumen that extends longitudinally through collet 562 (including flange 566 and stem 568). The lumen may also have an open distal end at a distal end of stem 568 (not shown). Opening 567 may be configured to receive distal portion 512 of vacuum tube 504. Vacuum tube 504 may be releasably coupled to connector 514 via collet 562. In examples, pushing a distal end of vacuum tube 504 distally into opening 567 of flange 566 of and through collet 562 (vacuum tube 504 may also interact with seals within housing member 570) may secure vacuum tube 504 to connector 514. In examples, pushing flange 566 of collet 562 in a distal direction (e.g., against housing member 570) may release vacuum tube 504 from connector 514 so that vacuum tube 504 may be pulled proximally and removed from the lumen of collet 562 via opening 567.

EVAC device 500 may further include a grasping portion 556. Grasping portion 556 may include a ring portion 558 (see FIG. 5B) configured to expand over housing member 570 of connector 514 (or another portion of connector 514) and over foam 502. Ring portion 558 may be flexible so that it can be stretched to position ring portion 558 over foam 502. When released, ring portion 558 may then compress over foam 502. Ring portion 558 may interact with a groove (not shown) formed on a radially outer surface of housing member 570 of connector 514 (or another portion of connector 514) within foam 502. Grasping portion 556 may include a tab 560 (e.g., an extension) extending from ring portion 558. Tab 560 may allow for a tool, such as grasper 142, to latch onto tab 560 to move foam 502 to a target site, as discussed above. In some examples, tab 560 may be made of a rigid elastomer. Alternatively, tab 560 (and other portions of grasping portion 556) may be formed of a flexible material.

Components of medical device 500 may be packaged in a kit. For example, a kit may include various sizes of foam 502 each coupled to a respective connector 514 to assist with treating target sites of varying sizes. For example, a target site will grow smaller as a wound at the target site starts to heal. Therefore, an initial foam used to treat the wound may not be an ideal size to treat the wound after a few days. The kit may allow a user to select a foam 502, coupled to a respective connector 514, of a size based on a location or size of a wound. A user may easily couple vacuum tube 504, along with a vacuum source, to foam 502 via connector 514 to treat a wound.

An exemplary method of using device 500 will now be described. A user may select foam 502, along with its respective connector 514, of a particular size based on size and location of a target site in the GI tract. Grasping portion 556 may be pre-secured to foam 502. Alternatively, a user may secure grasping portion 556 to foam 502 and/or connector 514 by placing ring portion 558 over foam 502 and positioning ring portion 558 within a groove on a radially outward surface of housing member 570 of connector 514 (or another portion of connector 514). Distal portion 512 of vacuum tube 504 may then be secured to connector 514 via collet 562. For example, a user may insert distal portion 512 of vacuum tube 504 into opening 567.

An endoscope may then be inserted into the GI tract via a natural orifice and foam 502 may be guided through a working channel of the endoscope to position foam 502 at or near the target site. A tool or other accessory device, such as grasper 142 (FIG. 1A-1B), may be routed through the working channel to grasp tab 560 of grasping portion 556 to position foam 502 at the target site. Negative pressure may then be supplied to vacuum tube 504 via a vacuum source to pull fluid, material, and/or other debris into the lumen of vacuum tube 504 via the openings of foam 502 to promote healing of the target site. The endoscope may be removed, and foam 502 and vacuum tube 504 may remain within a subject's body. In examples, vacuum tube 504 may be a nasogastric tube and may exit a subject's nose.

After a suitable amount of time (e.g., 3-5 days), the user may remove foam 502 (e.g., via a mouth of the subject), and detach connector 514, along with foam 502, from distal portion 512 of vacuum tube 504, as described above with respect to FIGS. 5A-5D. The user may then select another foam 502 (e.g., a foam 502 of a different size), along with its respective connector 514, based on properties (e.g., an adjusted size of the target site). Distal portion 512 of vacuum tube 504 may then be secured to connector 514 of the new foam 502 via collet 562 (as discussed above), and may then be ready for positioning at the target site. The user may utilize an endoscope to insert the new foam 502.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device including:
an implant having a plurality of openings;
a tube having a lumen extending therethrough;
a body extending from a distal portion of the tube to a proximal portion of the implant, such that the plurality of openings are in fluid communication with the lumen; and
a grasping portion configured to be grasped by a second medical device.

2. The medical device of claim 1, wherein the grasping portion includes at least one of a tab, a strap, or an opening.

3. The medical device of claim 1, wherein the tube is removably coupled to the body.

4. The medical device of claim 1, wherein a distal portion of the body is wider than a proximal portion of the body.

5. The medical device of claim 1, wherein a distal opening of the body is configured to receive the proximal portion of the implant, and wherein a proximal opening of the body is configured to receive the distal portion of the tube.

6. The medical device of claim 5, wherein a first portion of the distal opening is distal to a second portion of the distal opening.

7. The medical device of claim 5, wherein the body includes a lumen extending from the distal opening to the proximal opening.

8. The medical device of claim 1, wherein the proximal portion of the implant is compressed within the body.

9. The medical device of claim 1, wherein the tube is configured to couple to a vacuum source to supply a negative pressure to the implant.

10. The medical device of claim 1, wherein the grasping portion includes at least one opening formed on a radially outer surface of the body, and wherein the at least one opening is positioned adjacent to a distal opening of the body.

11. The medical device of claim 1, wherein the grasping portion includes at least one strap fixed to a radially outer surface of the body.

12. The medical device of claim 1, wherein the grasping portion includes at least one tab extending radially outward from the implant.

13. The medical device of claim 12, wherein a protruding portion of the at least one tab extends radially beyond a radially outer surface of the implant.

14. The medical device of claim 1, wherein the implant is disposed over a portion of the body, and wherein the grasping portion includes a ring positioned over the implant and the body.

15. The medical device of claim 14, wherein the grasping portion includes a tab extending from the ring.

16. A medical device including:
an implant having a plurality of openings;
a tube having a lumen extending therethrough and in fluid communication with the plurality of openings;
a body extending from a proximal end to a distal end, wherein the body tapers radially inward from the distal end to the proximal end, and wherein the body couples the implant to the tube; and
a grasping portion configured to be grasped by a second medical device.

17. The medical device of claim 16, wherein the distal end of the body is angled, and wherein a first portion of a distal opening of the body is distal to a second portion of the distal opening of the body.

18. The medical device of claim 17, wherein the grasping portion includes an opening formed on a radially outer surface of the body, and wherein the opening is adjacent to an outer edge of the distal opening.

19. A medical device including:
an implant having a plurality of openings;
a tube having a lumen extending therethrough and configured to couple to a vacuum source;
a body coupling the implant to the tube, such that the plurality of openings are in fluid communication with the lumen; and
a grasping portion configured to be grasped by a second medical device, wherein the grasping portion includes at least one of a tab, a strap, or an opening.

20. The medical device of claim 19, wherein the body includes the grasping portion.
